(19) United States
(12) Patent Application Publication
(10) Pub. No.: US 2023/0067215 A1
(43) Pub. Date: Mar. 2, 2023

(54) Gel for Injection Containing Controlled Degradation Polyester Microspheres

(71) Applicant: Hangzhou Singclean Medical Products Co., Ltd, Hangzhou (CN)

(72) Inventors: Jie Li, Hangzhou (CN); Yang Fu, Hangzhou (CN); Cuiying Zhou, Hangzhou (CN); Feifei Wu, Hangzhou (CN); Shitu Ma, Hangzhou (CN)

(21) Appl. No.: 17/539,154

(22) Filed: Nov. 30, 2021

(30) Foreign Application Priority Data
Aug. 24, 2021 (CN) 202110972080.7

(51) Int. Cl. A61K 8/04; A61K 8/02; A61K 8/85; A61K 8/73; A61K 8/92

(52) U.S. Cl. CPC A61K 8/042; A61K 8/025; A61K 8/85; A61K 8/735; A61K 8/92; A61Q 19/08

(57) ABSTRACT

The present invention provides a gel for injection containing controlled degradation polyester microspheres. The gel for injection consists of crosslinked sodium hyaluronate gel, non-crosslinked sodium hyaluronate gel, polyester microspheres wrapped with hard fat and a balanced salt solution. The polyester microspheres are separated from water by means of wrapping and hydrophobic effect of the hard fat, and accordingly the present invention solve the problem that polyester materials are prone to degradation in gel. In the meantime, the hard fat has the characteristic of low melting point (melting point being 33° C.-39° C.), after the product is injected into human body, the hard fat is molten and separated under the action of human body temperature, and the wrapped polyester microspheres are released so that the microspheres can be degraded in the human body to achieve controlled degradation.

### CROSS REFERENCE TO RELATED APPLICATION

The present application claims the benefit of Chinese Patent Application No. 202110972080.7 filed on Aug. 24, 2021, the contents of which are incorporated herein by reference in their entirety.

### FIELD OF THE INVENTION

The present invention relates to a gel for injection containing controlled degradation polyester microspheres, and belongs to the technical field of medical cosmetic materials.

### BACKGROUND OF THE INVENTION

With the development of medical cosmetology industry, injection materials for repairing facial wrinkles have attracted more and more attention. At present, these injection materials mainly fall into two categories. One is filling products represented by crosslinked sodium hyaluronate gel and collagen gel, such as Restylane® in the United States, Sunmax in Taiwan, etc. The main mechanism of action is subcutaneous filling, and after injection, the subcutaneous defects are expanded and filled to smooth the wrinkles. The other category is collagen stimulation products represented by poly-L-lactic acid (PLLA) microspheres, such as Sculptra™ in the United States. In the process of degradation, such products attract a large number of macrophages and other immune cells under the skin by stimulating the immune response of a host, resulting in the proliferation of fibroblasts and the differentiation of myofibroblasts to synthesize a large amount of collagen and accordingly restore the subcutaneous capacity and repairing the wrinkles.

The filling products can produce an immediate cosmetic effect after being injected, but are prone to being degraded under the action of enzymes in the body, and are thus short in duration. The products of PLLA microspheres are not affected by the enzymes and mainly experience degradation via gradual hydrolysis in the human body, so they have long maintenance time.

However, the effect of the PLLA microspheres needs to be produced gradually (it usually takes 1 to 3 months), which cannot meet the demand for immediate improvement of beauty lovers. Therefore, the two kinds of materials have their own advantages and disadvantages, a single material cannot fully meet the needs of the beauty lovers.

Chinese patent CN104258470A discloses polylactide microsphere and crosslinked hyaluronic acid mixed gel for injection and a preparation method thereof. Polylactide microspheres and crosslinked hyaluronic acid gel are mixed, and the obtained mixed gel allows instant filling for cosmetology and meanwhile lasts longer than pure sodium hyaluronate gel. However, studies found that the PLLA microspheres could not keep stable for a long time in an aqueous environment, and might have problems such as microsphere structure collapse or particle adhesion when preserved for more than one month.

### SUMMARY OF THE INVENTION

In view of the above defects of the prior art, the present invention provides a gel for injection containing controlled degradation polyester microspheres. When the gel for injection described in the present invention is not in use, the microspheres in the gel exhibit no degradation or micro-degradation at room temperature, and after use via subcutaneous injection, the polyester microspheres can be released under the action of human body temperature to accelerate the degradation of the microspheres in the body. For this purpose, the present invention adopts the following technical solution.

A gel for injection containing controlled degradation polyester microspheres is characterized in including crosslinked sodium hyaluronate gel, non-crosslinked sodium hyaluronate gel, polyester microspheres wrapped with hard fat and a balanced salt solution.

The microspheres in the gel for injection can keep stable at room temperature with no degradation or micro-degradation when not in use, and after subcutaneous injection for use, the polyester microspheres can be released under the action of human body temperature to accelerate the degradation of the microspheres in the body.

The controlled degradation characteristic described in the present invention is realized through the following principle:

When the gel for injection is prepared, the polyester microspheres wrapped with the hard fat are prepared firstly and named as first microspheres, then gel blocks of the crosslinked sodium hyaluronate gel wrapping outsides of the first microspheres are prepared, then the non-crosslinked sodium hyaluronate gel is added, and the mixture is stirred to obtain the gel for injection.

Further, preparation steps of the polyester microspheres wrapped with the hard fat include:

1) dissolving the hard fat in a solvent to prepare a solution of 0.5%-6%; and the solvent is preferably a volatile solvent, such as petroleum ether, ethyl ether, trichloromethane, etc;

2) under stirring, adding the polyester microspheres into the solution to make the microspheres evenly dispersed; and

3) conducting suction filtration to separate the microspheres from the solution, placing the microspheres into a room-temperature bellow for drying, and obtaining the polyester microspheres wrapped with the hard fat.

Wherein the hard fat includes one or more of type 34, type 36 and type 38 (with respective melting points of 33-35° C., 35-37° C. and 37-39° C.). Type 38 can be preferably used to improve stability of the product at room temperature.

The polyester microspheres include one or more of poly-L-lactic acid microspheres, polycaprolactone microspheres, glycolide-lactide copolymer microspheres and poly(p-dioxanone) microspheres and have a particle size range of 3-65 μm. The polyester microspheres are separated from water by means of the wrapping and hydrophobic effect of the hard fat, and accordingly the present invention solves the problem that polyester materials are prone to degradation and hard to preserve in gel. In the meantime, the hard fat has the characteristic of low melting point (melting point being 33° C.-39° C.), after the gel is injected into a human body, the hard fat is molten and separated under the action of human body temperature, and the wrapped polyester microspheres are released so that the microspheres can be degraded in the human body to achieve controlled degradation.

Further, the gel for injection containing the controlled degradation polyester microspheres is prepared through the following steps, and a method is simple in operation and easy to implement:

S1: preparing the polyester microspheres wrapped with the hard fat;

S2: dissolving sodium hyaluronate dry powder in purified water, then adding a crosslinking agent into the solution and stirring, then adding the polyester microspheres wrapped with the hard fat, stirring and reacting for 2-18 h to form the crosslinked sodium hyaluronate gel containing the microspheres; and divinyl sulphone or butanediol diglycidyl ether can be adopted as the crosslinking agent;

S3: breaking the above crosslinked sodium hyaluronate gel containing the microspheres into small gel blocks of 1-2 cm³, and then soaking and dialyzing the small gel blocks with the balanced salt solution;

S4: homogenizing the dialyzed gel with a dispersion machine, then adding the non-crosslinked sodium hyaluronate gel, and stirring the mixture for even dispersion; and

S5: conducting filling and sterilization to obtain a finished product.

The non-crosslinked sodium hyaluronate gel is prepared by dissolving the sodium hyaluronate dry powder in the balanced salt solution.

Preferably:

The feeding amount of the polyester microspheres wrapped with the hard fat accounts for 3%-30% of the total mass of the product;

the feeding amount of sodium hyaluronate accounts for, by dry powder, 1.2%-2.5% of the total mass of the product; and

the balanced salt solution is a sodium chloride solution or phosphate buffered solution with an osmotic pressure of 200-400 mOsmol/L and pH of 6.5-7.5.

The gel for injection containing the controlled degradation polyester microspheres of the present invention can improve the subcutaneous capacity of human skin and repair wrinkles, folds, scars and defer aging of the skin. The gel for injection containing the controlled degradation polyester microspheres provided by the present invention can immediately fill the subcutaneous tissue and smooth the wrinkles under the action of the crosslinked sodium hyaluronate gel after being injected and implanted; and after 3-6 months, while the crosslinked sodium hyaluronate gel is gradually degraded and absorbed, the gradually-degraded polyester microspheres can stimulate the subcutaneous immune response to enable autologous generation of collagen, and the filling is continued to a point where the subcutaneous capacity is maintained for 12-20 months.

To sum up, the present invention has the following excellent properties:

(1) the present invention prepares the polyester microspheres wrapped with the hard fat, the polyester microspheres are separated from the water by means of the wrapping and hydrophobic effect of the hard fat, so that the microspheres exhibit no degradation or micro-degradation in the gel at room temperature, thereby solving the problem that polyester materials are prone to degradation and hard to preserve in the gel.

(2) after the product provided by the present invention is injected into the human body, the hard fat is molten and separated under the action of human body temperature, and the wrapped polyester microspheres are released so that the microspheres can be degraded in the human body to achieve controlled degradation.

(3) the product provided by the present invention can immediately fill the subcutaneous tissue and smooth the wrinkles after being injected and implanted, subsequently, the gradually-degraded polyester microspheres can stimulate the subcutaneous immune response to enable autologous generation of collagen, and the filling is continued to a point where the subcutaneous capacity is maintained for 12-20 months, so that the long-time cosmetic effect is realized.

### BRIEF DESCRIPTION OF DRAWINGS

FIG. 1 and FIG. 2 are scanning electron microscope comparison diagrams of PLLA microspheres wrapped with hard fat (type 38) and unwrapped PLLA microspheres prepared by embodiment 1 of the present invention; wherein FIG. 1 shows the PLLA microspheres wrapped with hard fat (type 38), and FIG. 2 shows the unwrapped PLLA microspheres.

FIG. 3 is a degradation curve comparison diagram of the PLLA microspheres of a product prepared by embodiment 1 of the present invention and a control product at 25° C.

FIG. 4 is a degradation curve diagram of the PLLA microspheres of the product prepared by the embodiment 1 of the present invention at different storage temperatures.

### DETAILED DESCRIPTION

The substantive features and notable progress of the present invention are further clarified by introducing embodiments of the present invention, but the present invention is by no means limited to the embodiments.

### Embodiment 1

S1: PLLA microspheres wrapped with hard fat were prepared: the hard fat of type 38 was dissolved in petroleum ether to prepare a solution of 2%; under stirring, the PLLA microspheres were added into the solution to be evenly dispersed; suction filtration was conducted to separate the microspheres from the solution, the microspheres were placed into a room-temperature bellow to be dried, to obtain the PLLA microspheres wrapped with the hard fat;

S2: 10.0 g sodium hyaluronate (dry powder) was dissolved in 10 mL purified water, then 120 μL divinyl sulphone, as a crosslinking agent, was added into the solution and stirred for even mixing, then 30.5 g PLLA microspheres wrapped with the hard fat were added, the mixture was evenly stirred and allowed to react for 2-18 h to form crosslinked sodium hyaluronate gel containing the microspheres;

S3: the above gel was broken into small gel blocks of 1-2 cm³, and then the small gel blocks were soaked and dialyzed with the balanced salt solution;

S4: the dialyzed gel was homogenized with a dispersion machine, then the non-crosslinked sodium hyaluronate gel (prepared by dissolving sodium hyaluronate dry powder in the balanced salt solution) accounting for 10% of the mass of the dialyzed gel was added, and the mixture was stirred to be evenly dispersed; and

S5: filling and sterilization were conducted to obtain a finished product.

Gel containing PLLA microspheres unwrapped with the hard fat was prepared through the same steps as S2 to S5, serving as a control group.

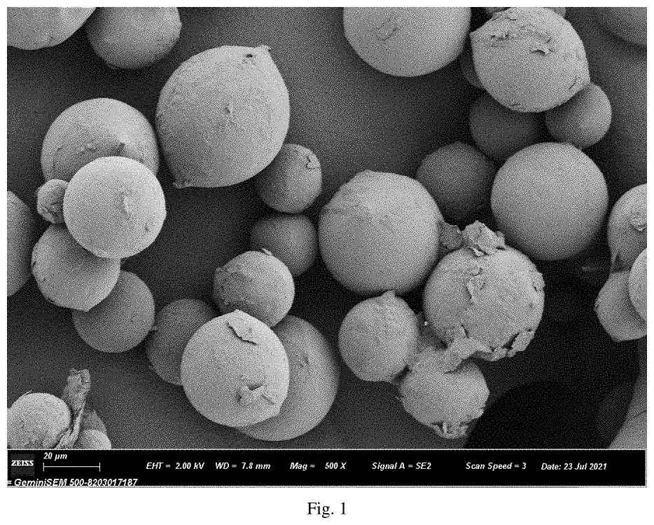

FIG. 1 shows the micromorphology of the PLLA microspheres wrapped with the hard fat (type 38), and when compared with the unwrapped microspheres in FIG. 2, it is obvious that a layer of rough hard fat was adhered to the smooth surface of the microspheres after treatment in S1.

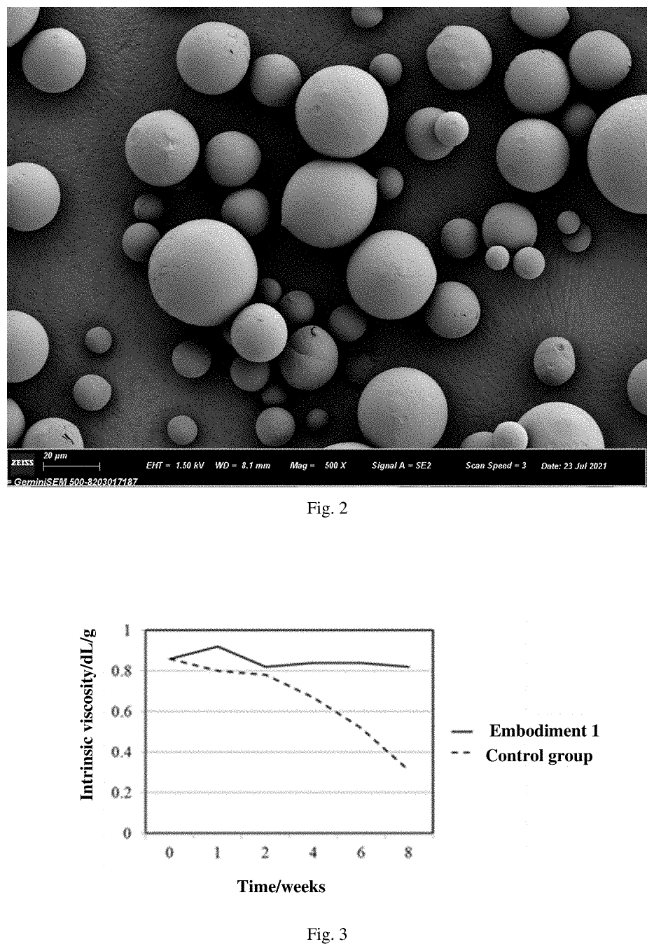

FIG. 3 shows the comparison of degradation rates of the gel containing the PLLA microspheres wrapped with the hard fat (this embodiment) and the gel containing the microspheres unwrapped with the hard fat (the control group) at room temperature (25° C.). By dissociating the gel with hyaluronidase, the PLLA microspheres were extracted from the two groups of gel, and then the intrinsic viscosity of the microspheres was measured through a Ubbelohde viscometer. As can be seen from the figure, at room temperature, the PLLA microspheres in the gel of this embodiment merely underwent a minor reduction in the intrinsic viscosity within 8 weeks, which indicated that there was little PLLA molecular degradation; and the PLLA microspheres in the gel of the control group underwent obvious degradation after preservation for 2 weeks, and degraded to a state of low intrinsic viscosity in the 8th week.

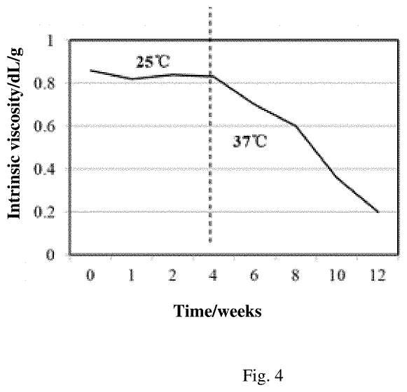

FIG. 4 shows a degradation curve diagram of the prepared gel preserved at 25° C. for 4 weeks, and then preserved at 37° C. for 12 weeks. As can be seen from the figure, the PLLA microspheres were hardly degraded in the gel within 4 weeks at a room-temperature preservation condition of 25° C.; while when the temperature rose to 37° C., the degradation was obvious, which reflected the controllable degradation characteristic of the PLLA microspheres in the sample.

### Embodiment 2

S1: polycaprolactone (PCL) microspheres wrapped with the hard fat were prepared: hard fat of type 36 was dissolved in trichloromethane to prepare a solution of 6%; under stirring, the PCL microspheres were added into the solution to be evenly dispersed; suction filtration was conducted to separate the microspheres from the solution, the microspheres were placed into a room-temperature bellow to be dried to obtain the PCL microspheres wrapped with the hard fat;

S2: 10.0 g sodium hyaluronate (dry powder) was dissolved in 100 mL purified water, then 120 μL divinyl sulphone as crosslinking agent was added into the solution to be stirred for even mixing, then 11.4 g PCL microspheres wrapped with the hard fat were added, and the mixture was evenly stirred and allowed to react for 2-18 h to form crosslinked sodium hyaluronate gel containing the microspheres;

S3: the above gel was broken into small gel blocks of 1-2 cm³, and then the small gel blocks were soaked and dialyzed with the balanced salt solution;

S4: the dialyzed gel was homogenized with a dispersion machine, then the non-crosslinked sodium hyaluronate gel (prepared by dissolving sodium hyaluronate dry powder in the balanced salt solution) accounting for 10% of the mass of the dialyzed gel was added, and the mixture was stirred to be evenly dispersed; and

S5: filling and sterilization were conducted to obtain a finished product.

### Embodiment 3

S1: PLLA microspheres wrapped with the hard fat were prepared: the hard fat of type 37 was dissolved in ether to prepare a solution of 0.5%; under stirring, the PLLA microspheres were added into the solution to be evenly dispersed; suction filtration was conducted to separate the microspheres from the solution, the microspheres were placed into a room-temperature bellow to be dried, to obtain the PLLA microspheres wrapped with the hard fat;

S2: 10.0 g sodium hyaluronate (dry powder)was dissolved in 100 mL purified water, then 120 μL divinyl sulphone as a crosslinking agent was added into the solution and stirred for even mixing, then 51.0 g PCL microspheres wrapped with the hard fat were added, and the mixture was evenly stirred and allowed to react for 2-18 h to form crosslinked sodium hyaluronate gel containing the microspheres;

S3: the above gel was broken into small gel blocks of 1-2 cm³, and then the small gel blocks were soaked and dialyzed with the balanced salt solution;

S4: the dialyzed gel was homogenized with a dispersion machine, then the non-crosslinked sodium hyaluronate gel (prepared by dissolving sodium hyaluronate dry powder in the balanced salt solution) accounting for 10% of the mass of the dialyzed gel was added, and a mixture was stirred for even dispersion; and

S5: filling and sterilization were conducted to obtain a finished product.

Those skilled in the art will easily understood that the above mentioned are only preferable embodiments of the present invention and shall not be used to limit the present invention. Any modification, equivalent substitution or improvement made within the spirit and principles of the present invention shall be included in the scope of protection of the present invention.

1. A gel for injection containing controlled degradation polyester microspheres, comprising crosslinked sodium hyaluronate gel, non-crosslinked sodium hyaluronate gel, polyester microspheres wrapped with hard fat and a balanced salt solution.

2. The gel for injection containing the controlled degradation polyester microspheres of claim 1, wherein the microspheres in the gel for injection remain stable at room temperature with no degradation or micro-degradation when not in use, and after use via subcutaneous injection, the polyester microspheres are released under the action of human body temperature to accelerate degradation of the micro spheres in the body.

3. The gel for injection containing the controlled degradation polyester microspheres of claim 1, wherein when the gel for injection is prepared, the polyester microspheres wrapped with the hard fat are prepared firstly and named as first microspheres, then gel blocks of the crosslinked sodium hyaluronate gel wrapping outsides of the first microspheres are prepared, then the non-crosslinked sodium hyaluronate gel is added to obtain a mixture which is stirred for even dispersion, and the gel for injection is obtained.

4. The gel for injection containing the controlled degradation polyester microspheres of claim 3, wherein preparation steps of the polyester microspheres wrapped with the hard fat comprise:
1) dissolving the hard fat in a solvent to prepare a solution of 0.5%-6%;
2) under stirring, adding the polyester microspheres into the solution to make the microspheres evenly dispersed; and
3) conducting suction filtration to separate the microspheres from the solution, placing the microspheres into a room-temperature bellow for drying, and obtaining the polyester microspheres wrapped with the hard fat.

5. The gel for injection containing the controlled degradation polyester microspheres of claim 1, wherein the hard fat comprises one or more of type 34, type 36 and type 38.

6. The gel for injection containing the controlled degradation polyester microspheres of claim 1, wherein the polyester microspheres comprise one or more of poly-L-lactic acid microspheres, p olycaprolactone microspheres, glycolide-lactide copolymer microspheres and poly(p-dioxanone) microspheres and have a particle size range of 3-65 μm.

7. The gel for injection containing the controlled degradation polyester microspheres of claim 3, characterized in being prepared through the following steps:
S1: preparing the polyester microspheres wrapped with the hard fat;
S2: dissolving sodium hyaluronate dry powder in purified water, adding a crosslinking agent in the solution, stirring, adding the polyester microspheres wrapped with the hard fat, stirring and reacting for 2-18 h to form the crosslinked sodium hyaluronate gel containing the microspheres;
S3: breaking the above crosslinked sodium hyaluronate gel containing the microspheres into small gel blocks of 1-2 cm³, and soaking and dialyzing the small gel blocks with the balanced salt solution;
S4: homogenizing the dialyzed gel with a dispersion machine, adding the non-crosslinked sodium hyaluronate gel, and stirring; and
S5: conducting filling and sterilization to obtain a finished product.

8. The gel for injection containing the controlled degradation polyester microspheres of claim 7, wherein feeding amount of the polyester microspheres wrapped with the hard fat accounts for 3%-30% of total mass of a product; and feeding amount of sodium hyaluronate accounts for, by dry powder, 1.2%-2.5% of the total mass of the product; and
the balanced salt solution is a sodium chloride solution or phosphate buffered solution with an osmotic pressure of 200-400 mOsmol/L and pH of 6.5-7.5.

9. The gel for injection containing the controlled degradation polyester microspheres of claim 7, wherein the non-crosslinked sodium hyaluronate gel is prepared by dissolving the sodium hyaluronate dry powder in the balanced salt solution.

10. The gel for injection containing the controlled degradation polyester microspheres of claim 1, wherein the gel is capable of improving subcutaneous capacity of human skin and repairing wrinkles, folds, scars and deferring aging of the skin.
